(19) United States
(12) Patent Application Publication
(10) Pub. No.: US 2011/0275711 A1
(43) Pub. Date: Nov. 10, 2011

(54) METHOD FOR TREATING MACULAR EDEMA

(75) Inventors: Yukihiko MASHIMA, Tokyo-to (JP); Ryuji UENO, Montgomery, MD (US)

(73) Assignee: R-TECH UENO, LTD., Tokyo (JP)

(21) Appl. No.: 13/084,982

(22) Filed: Apr. 12, 2011

Related U.S. Application Data
(60) Provisional application No. 61/323,338, filed on Apr. 12, 2010.
(60) Provisional application No. 61/323,342, filed on Apr. 12, 2010.
(60) Provisional application No. 61/326,811, filed on Apr. 22, 2010.
(60) Provisional application No. 61/362,945, filed on Jul. 9, 2010.
(60) Provisional application No. 61/408,237, filed on Oct. 29, 2010.

(51) Int. Cl. A61K 31/215; A61P 27/02

(52) U.S. Cl. 514/530

(57) ABSTRACT

Provided is a method for treating macular edema in a mammalian subject, comprising administering an effective amount of a fatty acid derivative to the subject in need thereof. The method of the present invention can effectively treat macular edema in a non-invasive manner. In one embodiment of the present invention, the fatty acid derivative is isopropyl unoprostone.

### CROSS REFERENCES OF RELATED APPLICATIONS

This application claims the benefit of U.S. Provisional Application Nos. 61/323,338 filed Apr. 12, 2010, 61/323,342 filed Apr. 12, 2010, 61/326,811 filed Apr. 22, 2010, 61/362,945 filed Jul. 9, 2010 and 61/408,237 filed Oct. 29, 2010. The contents of those provisional applications are herein incorporated by reference.

### BACKGROUND OF THE INVENTION

### Field of the Invention

The present invention relates to a pharmaceutical composition and a method for treating macular edema in a mammalian subject.

Macular edema is an eye condition characterized by a buildup of fluid in the macula of the eye. This condition may occur when blood vessels in the retina leaks fluid, allowing fluid to build up in the macula. This fluid contains water, fat and the like and causes the macula to swell and thicken. According to the degree of edema, the patient comes down subjective symptoms such as vision loss, metamorphopsia (things look distorted things), and micropsia (things look smaller things). Various conditions or disease cause macular edema. It has been known that there are diabetic macular edema and non-diabetic macular edema. The macular is a small area of the retina responsible for approximately the central 15 degrees of the visual field and plays an important role on control of the vision. Once the macula edema occurs, patient's vision will be damaged over the progress of the condition. If the condition is not treated, macular edema may cause permanent vision loss due to the irreversible change of the macular. In addition, it has also been suggested that macular edema may accelerate the progress of retinopathy.

Known treatments of macular edema include symptomatic treatment such as laser photocoagulation and surgical operation of the vitreous body, and drug treatment such as direct injection of a steroid into the eyes, for example, under the conjunctiva. However, laser irradiation precisely to the macular is not easy and may cause inflammatory that results in further progress of the edema. Surgical operation of the vitreous body causes severe physical and economical burden on the patient. In addition, the treatment cannot suppress recurrence of the macular edema. Direct ocular injection of a steroid causes severe physical and mental burden to the patient and may accompany with side effects such as elevation of the intraocular pressure.

Fatty acid derivative are members of class of organic carboxylic acids, which are contained in tissues or organs of human and other mammals, and exhibit a wide range of physiological activities. Some fatty acid derivatives found in nature have, as a general structural property thereof, a prostanoic acid skeleton as shown in the formula (A):

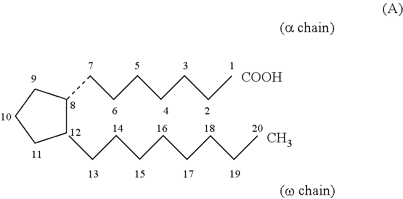

On the other hand, some synthetic Prostaglandin (PG) analogues have modified skeletons. The primary PGs are classified into PGAs, PGBs, PGCs, PGDs, PGEs, PGFs, PGGs, PGHs, PGIs and PGJs on the basis of the structural property of the five membered ring moiety, and further classified into the following three types by the number and position of the unsaturated bond in the carbon chain moiety.
Type 1 (subscript 1): 13,14-unsaturated-15-OH
Type 2 (subscript 2): 5,6- and 13,14-diunsaturated-15-OH
Type 3 (subscript 3): 5,6-, 13,14-, and 17,18-triunsaturated-15-OH.

Further, PGFs are classified on the basis of the configuration of the hydroxy group at the 9-position into a type (wherein the hydroxy group is of the α-configuration) and β type (wherein the hydroxy group is of the β-configuration).

Prostones, having an oxo group at position 15 of prostanoic acid skeleton (15-keto type) and having a single bond between positions 13 and 14 and an oxo group at position 15 (13,14-dihydro-15-keto type), are fatty acid derivatives known as substances naturally produced by enzymatic actions during metabolism of the primary PGs and have some therapeutic effect. Prostones have been disclosed in U.S. Pat. Nos. 5,073,569, 5,534,547, 5,225,439, 5,166,174, 5,428,062 5,380,709 5,886,034 6,265,440, 5,106,869, 5,221,763, 5,591,887, 5,770,759 and 5,739,161, the contents of these references are herein incorporated by reference.

Some fatty acid derivatives have been known as drugs used in the ophthalmic field, for example, for lowering intraocular pressure or treating glaucoma. For example, (+)-Isopropyl (Z)-7-[(1R,2R,3R,5S)-3,5-dihydroxy-2-[(3R)-3-hydroxy-5-phenylpentyl]cyclopentyl]-5-heptenoate (general name: latanoprost), Isopropyl (5Z)-7-((1R,2R,3R,5S)-3,5-dihydroxy-2-{(1E,3R)-3-hydroxy-4-[3-(trifluoromethyl)phenoxy]but-1-enyl}cyclopentyl)hept-5-enoate (general name: travoprost), (5Z)-7-{(1R,2R,3R,5S)-3,5-Dihydroxy-2-[(1E,3S)-3-hydroxy-5-phenylpent-1-en-1-yl]cyclopentyl}-N-ethylhept-5-enamide (general name: bimatoprost) and 1-Methylethyl(5Z)-7-{(1R,2R,3R,5S)-2-[(1E)-3,3-difluoro-4-phenoxy-1-butenyl]-3,5-dihydroxy cyclopentyl}-5-heptenoate (general name: tafluprost) have been marketed as ophthalmic solution for the treatment of glaucoma and/or ocular hypertension under the name of Xalatan®, Travatan®, Lumigan® and Tapros®, respectively.

It has been known that latanoprost, which is a fatty acid derivative having a hydroxy group at position 15 of prostanoic acid skeleton, causes macular edema as side effect. See package insert of Xalatan®.

On the other hand, Prostones have also been known to be useful in the ophthalmic field, for example, for lowering intraocular pressure and treating glaucoma (U.S. Pat. Nos. 5,001,153, 5,151,444, 5,166,178, 5,194,429 and 5,236,907), for treating cataract (U.S. Pat. Nos. 5,212,324 and 5,686,487), for increasing the choroidal blood flow (U.S. Pat. No. 5,221,690), for treating optic nerve disorder (U.S. Pat. No. 5,773,471), the contents of these references are herein incorporated by reference. Ophthalmic solution comprising (+)-isopropyl (Z)-7-[(1R,2R,3R,5S)-3,5-dihydroxy-2-(3-oxodecyl)cyclopentyl]hept-5-enoate (general name: isopropyl unoprostone) has been marketed under the name of Rescula® as a pharmaceutical product for the treatment of glaucoma and ocular hypertension.

In view of the burden on the patient, the treatment of ophthalmic diseases is desirably effected by noninvasively administering a drug, for example, by instilling an eye drop to the eyes. However, as discussed above, no satisfying treatment for macular edema has been known to date.

### DISCLOSURE OF THE INVENTION

### Problem to be Solved by the Invention

An object of the present invention to provide a pharmaceutical composition and method for treating macular edema, particularly, for treating macular edema by non invasive way.

### SUMMARY OF THE INVENTION

The present inventors have found that a specific fatty acid derivative can effectively treat macular edema by administering the same in non invasive way to the patient in need thereof, and completed the invention.

Accordingly, the present application provide the following invention:

(1) A pharmaceutical composition comprising a fatty acid derivative represented by the formula (I):

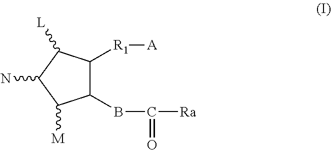

wherein L, M and N are hydrogen, hydroxy, halogen, lower alkyl, hydroxy(lower)alkyl, lower alkanoyloxy or oxo, wherein at least one of L and M is a group other than hydrogen and the five-membered ring may have at least one double bond;

A is —CH₃, —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;

R₁ is saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and

Ra is saturated or unsaturated lower or medium bivalent aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; or heterocyclic-oxy group; as an active ingredient for treating macular edema in a mammalian subject.

(2) The composition of (1), wherein Ra is a hydrocarbon containing 6-10 carbon atoms.

(3) The composition of (2), wherein Ra is a hydrocarbon containing 7 carbon atoms.

(4) The composition of (1), wherein L is hydroxy, M is hydrogen and N is hydroxy.

(5) The composition of (4), wherein R₁ is —CH₂—CH═CH—CH₂—CH₂—CH₂— and Ra is a hydrocarbon containing 7 carbon atoms.

(6) The composition of (1), wherein B is —CH₂—CH₂—.

(7) The composition of (6), wherein the fatty acid derivative is isopropyl unoprostone.

(8) The composition of (1), which is for topical ophthalmic administration.

(9) The composition of (8), which is an ophthalmic solution.

(10) A method for treating macular edema in a mammalian subject, comprising administering an effective amount of the fatty acid derivative represented by the formula (I) to the subject in need thereof.

(11) Use of the fatty acid derivative represented by the formula (I) for manufacturing a pharmaceutical composition for treating macular edema in a mammalian subject.

### DETAILED DISCLOSURE OF THE INVENTION

The nomenclature of the fatty acid derivative used herein is based on the numbering system of prostanoic acid represented in the above formula (A).

The formula (A) shows a basic skeleton of the C-20 carbon atoms fatty acid derivative, but the present invention is not limited to those having the same number of carbon atoms. In the formula (A), the numbering of the carbon atoms which constitute the basic skeleton of the prostanoic acid starts at the carboxylic acid (numbered 1), and carbon atoms in the α-chain are numbered 2 to 7 towards the five-membered ring, those in the ring are 8 to 12, and those in the ω-chain are 13 to 20. When the number of carbon atoms is decreased in the α-chain, the number is deleted in the order starting from position 2; and when the number of carbon atoms is increased in the α-chain, compounds are named as substitution compounds having respective substituents at position 2 in place of carboxy group (C-1). Similarly, when the number of carbon atoms is decreased in the w-chain, the number is deleted in the order starting from position 20; and when the number of carbon atoms is increased in the ω-chain, the carbon atoms at the position 21 or later are named as a substituent at position 20. Stereochemistry of the compounds is the same as that of the above formula (A) unless otherwise specified.

In general, each of PGD, PGE and PGF represents a fatty acid derivative having hydroxy groups at positions 9 and/or 11, but in the present specification they also include those having substituents other than the hydroxy groups at positions 9 and/or 11. Such compounds are referred to as 9-deoxy-9-substituted-fatty acid derivatives or 11-deoxy-11-substituted-fatty acid derivatives. A fatty acid derivative having hydrogen in place of the hydroxy group is simply named as 9- or 11-deoxy-fatty acid derivative.

As stated above, the nomenclature of a fatty acid derivative is based on the prostanoic acid skeleton. In the case the compound has similar partial structure as the primary PG, the abbreviation of “PG” may be used. Thus, a fatty acid derivative whose α-chain is extended by two carbon atoms, that is, having 9 carbon atoms in the α-chain is named as 2-decarboxy-2-(2-carboxyethyl)-PG compound. Similarly, a fatty acid derivative having 11 carbon atoms in the α-chain is named as 2-decarboxy-2-(4-carboxybutyl)-PG compound. Further, a fatty acid derivative whose ω-chain is extended by two carbon atoms, that is, having 10 carbon atoms in the ω-chain is named as 20-ethyl-PG compound. These compounds, however, may also be named according to the IUPAC nomenclatures and the like.

Examples of the analogues including substitution compounds or derivatives of the above described fatty acid derivative include a fatty acid derivative whose carboxy group at the end of the alpha chain is esterified; a fatty acid derivative whose a chain is extended, a physiologically acceptable salt thereof, a fatty acid derivative having a double bond between positions 2 and 3 or a triple bond between positions 5 and 6; a fatty acid derivative having substituent(s) on carbon atom(s) at position(s) 3, 5, 6, 16, 17, 18, 19 and/or 20; and a fatty acid derivative having a lower alkyl or a hydroxy (lower) alkyl group at position 9 and/or 11 in place of the hydroxy group.

According to the present invention, preferred substituents on the carbon atom at position(s) 3, 17, 18 and/or 19 include alkyl having 1-4 carbon atoms, especially methyl and ethyl. Preferred substituents on the carbon atom at position 16 include lower alkyls such as methyl and ethyl, hydroxy, halogen atom such as chlorine and fluorine, and aryloxy such as trifluoromethylphenoxy. Preferred substituents on the carbon atom at position 17 include lower alkyl such as methyl and ethyl, hydroxy, halogen atom such as chlorine and fluorine, and aryloxy such as trifluoromethylphenoxy. Preferred substituents on the carbon atom at position 20 include saturated or unsaturated lower alkyl such as C₁₋₄ alkyl, lower alkoxy such as C₁₋₄ alkoxy, and lower alkoxy alkyl such as C₁₋₄ alkoxy-C₁₋₄ alkyl. Preferred substituents on the carbon atom at position 5 include halogen atoms such as chlorine and fluorine. Preferred substituents on the carbon atom at position 6 include an oxo group forming a carbonyl group. Stereochemistry of PGs having hydroxy, lower alkyl or hydroxy(lower)alkyl substituent on the carbon atom at positions 9 and 11 may be α, β or a mixture thereof.

Further, the above described analogues or derivatives may have an ω-chain shorter than that of the primary PGs and a substituent such as alkoxy, cycloalkyl, cycloalkyloxy, phenoxy and phenyl at the end of the truncated ω-chain.

The fatty acid derivative used in the instant application is represented by the formula (I):

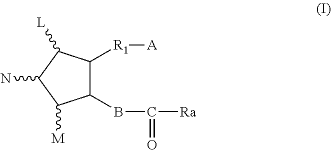

wherein L, M and N are hydrogen, hydroxy, halogen, lower alkyl, hydroxy(lower)alkyl, lower alkanoyloxy or oxo, wherein at least one of L and M is a group other than hydrogen and the five-membered ring may have at least one double bond;

A is —CH₃, —CH₂OH, —COCH₂OH, —COOH or a functional derivative thereof;

B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;

R₁ is saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and

Ra is saturated or unsaturated lower or medium bivalent aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; or heterocyclic-oxy group.

A more preferred fatty acid derivative used in the present invention is represented by the formula (II):

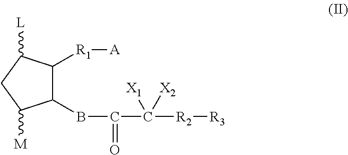

wherein L and M are hydrogen, hydroxy, halogen, lower alkyl, hydroxy(lower)alkyl, lower alkanoyloxy or oxo, wherein at least one of L and M is a group other than hydrogen and the five-membered ring may have at least one double bond;

A is —CH₃, —CH₂OH, —COCH₂OH, —COOH or a functional derivative thereof;

B is single bond, —CH₂—CH₂—, —CH═CH—, —C≡—, —CH₂—CH₂—CH₂—, —CH═CH—CH₂—, —CH₂—CH═CH—, —C≡C—CH₂— or —CH₂—C≡C—;

X₁ and X₂ are hydrogen, lower alkyl, or halogen; R₁ is saturated or unsaturated bivalent lower or medium aliphatic hydrocarbon residue, which is unsubstituted or substituted with halogen, lower alkyl, hydroxy, oxo, aryl or heterocyclic group, and at least one carbon atom in the aliphatic hydrocarbon is optionally substituted by oxygen, nitrogen or sulfur;

R₂ is single bond or lower alkylene; and

R₃ is lower alkyl, lower alkoxy, lower alkanoyloxy, cyclo(lower)alkyl, cyclo(lower)alkyloxy, aryl, aryloxy, heterocyclic group or heterocyclic-oxy group.

In the above formula, the term “unsaturated” in the definitions for R₁ and Ra is intended to include at least one or more double bonds and/or triple bonds that are isolatedly, separately or serially present between carbon atoms of the main and/or side chains. According to the usual nomenclature, an unsaturated bond between two serial positions is represented by denoting the lower number of the two positions, and an unsaturated bond between two distal positions is represented by denoting both of the positions.

The term “lower or medium aliphatic hydrocarbon” refers to a straight or branched chain hydrocarbon group having 1 to 14 carbon atoms (for a side chain, 1 to 3 carbon atoms are preferable) and preferably 1 to 10, especially 1 to 8 carbon atoms.

The term “halogen atom” covers fluorine, chlorine, bromine and iodine.

The term “lower” throughout the specification is intended to include a group having 1 to 6 carbon atoms unless otherwise specified.

The term “lower alkyl” refers to a straight or branched chain saturated hydrocarbon group containing 1 to 6 carbon atoms and includes, for example, methyl, ethyl, propyl, isopropyl, butyl, isobutyl, t-butyl, pentyl and hexyl.

The term “lower alkylene” refers to a straight or branched chain bivalent saturated hydrocarbon group containing 1 to 6 carbon atoms and includes, for example, methylene, ethylene, propylene, isopropylene, butylene, isobutylene, t-butylene, pentylene and hexylene.

The term “lower alkoxy” refers to a group of lower alkyl-O—, wherein lower alkyl is as defined above.

The term “hydroxy(lower)alkyl” refers to a lower alkyl as defined above which is substituted with at least one hydroxy group such as hydroxymethyl, 1-hydroxyethyl, 2-hydroxyethyl and 1-methyl-1-hydroxyethyl.

The term “lower alkanoyloxy” refers to a group represented by the formula RCO—O—, wherein RCO— is an acyl group formed by oxidation of a lower alkyl group as defined above, such as acetyl.

The term “cyclo(lower)alkyl” refers to a cyclic group formed by cyclization of a lower alkyl group as defined above but contains three or more carbon atoms, and includes, for example, cyclopropyl, cyclobutyl, cyclopentyl and cyclohexyl.

The term “cyclo(lower)alkyloxy” refers to the group of cyclo(lower)alkyl-O—, wherein cyclo(lower)alkyl is as defined above.

The term “aryl” may include unsubstituted or substituted aromatic hydrocarbon rings (preferably monocyclic groups), for example, phenyl, tolyl, xylyl. Examples of the substituents are halogen atom and halo(lower)alkyl, wherein halogen atom and lower alkyl are as defined above.

The term “aryloxy” refers to a group represented by the formula ArO—, wherein Ar is aryl as defined above.

The term “heterocyclic group” may include mono- to tri-cyclic, preferably monocyclic heterocyclic group which is 5 to 14, preferably 5 to 10 membered ring having optionally substituted carbon atom and 1 to 4, preferably 1 to 3 of 1 or 2 type of hetero atoms selected from nitrogen atom, oxygen atom and sulfur atom. Examples of the heterocyclic group include furyl, thienyl, pyrrolyl, oxazolyl, isoxazolyl, thiazolyl, isothiazolyl, imidazolyl, pyrazolyl, furazanyl, pyranyl, pyridyl, pyridazinyl, pyrimidyl, pyrazinyl, 2-pyrrolinyl, pyrrolidinyl, 2-imidazolinyl, imidazolidinyl, 2-pyrazolinyl, pyrazolidinyl, piperidino, piperazinyl, morpholino, indolyl, benzothienyl, quinolyl, isoquinolyl, purinyl, quinazolinyl, carbazolyl, acridinyl, phenanthridinyl, benzimidazolyl, benzimidazolinyl, benzothiazolyl, phenothiazinyl. Examples of the substituent in this case include halogen, and halogen substituted lower alkyl group, wherein halogen atom and lower alkyl group are as described above.

The term “heterocyclic-oxy group” means a group represented by the formula HcO—, wherein Hc is a heterocyclic group as described above.

The term “functional derivative” of A includes salts, preferably pharmaceutically acceptable salts, ethers, esters and amides.

Suitable “pharmaceutically acceptable salts” include salts formed with non-toxic bases conventionally used in pharmaceutical field, for example a salt with an inorganic base such as an alkali metal salt (such as sodium salt and potassium salt), an alkaline earth metal salt (such as calcium salt and magnesium salt), an ammonium salt; or a salt with an organic base, for example, an amine salt (such as methylamine salt, dimethylamine salt, cyclohexylamine salt, benzylamine salt, piperidine salt, ethylenediamine salt, ethanolamine salt, diethanolamine salt, triethanolamine salt, tris(hydroxymethylamino)ethane salt, monomethyl-monoethanolamine salt, procaine salt and caffeine salt), a basic amino acid salt (such as arginine salt and lysine salt), tetraalkyl ammonium salt and the like. These salts may be prepared by a conventional process, for example from the corresponding acid and base or by salt interchange.

Examples of the ethers include alkyl ethers, for example, lower alkyl ethers such as methyl ether, ethyl ether, propyl ether, isopropyl ether, butyl ether, isobutyl ether, t-butyl ether, pentyl ether and 1-cyclopropyl ethyl ether; and medium or higher alkyl ethers such as octyl ether, diethylhexyl ether, lauryl ether and cetyl ether; unsaturated ethers such as oleyl ether and linolenyl ether; lower alkenyl ethers such as vinyl ether, allyl ether; lower alkynyl ethers such as ethynyl ether and propynyl ether; hydroxy(lower)alkyl ethers such as hydroxyethyl ether and hydroxyisopropyl ether; lower alkoxy (lower)alkyl ethers such as methoxymethyl ether and 1-methoxyethyl ether; optionally substituted aryl ethers such as phenyl ether, tosyl ether, t-butylphenyl ether, salicyl ether, 3,4-di-methoxyphenyl ether and benzamidophenyl ether; and aryl(lower)alkyl ethers such as benzyl ether, trityl ether and benzhydryl ether.

Examples of the esters include aliphatic esters, for example, lower alkyl esters such as methyl ester, ethyl ester, propyl ester, isopropyl ester, butyl ester, isobutyl ester, t-butyl ester, pentyl ester and 1-cyclopropylethyl ester; lower alkenyl esters such as vinyl ester and allyl ester; lower alkynyl esters such as ethynyl ester and propynyl ester; hydroxy(lower)alkyl ester such as hydroxyethyl ester; lower alkoxy (lower) alkyl esters such as methoxymethyl ester and 1-methoxyethyl ester; optionally substituted aryl esters such as, for example, phenyl ester, tolyl ester, t-butylphenyl ester, salicyl ester, 3,4-di-methoxyphenyl ester and benzamidophenyl ester; and aryl(lower)alkyl ester such as benzyl ester, trityl ester and benzhydryl ester.

The amide of A means a group represented by the formula —CONR′R″, wherein each of R′ and R″ is hydrogen, lower alkyl, aryl, alkyl- or aryl-sulfonyl, lower alkenyl and lower alkynyl, and include for example lower alkyl amides such as methylamide, ethylamide, dimethylamide and diethylamide; arylamides such as anilide and toluidide; and alkyl- or aryl-sulfonylamides such as methylsulfonylamide, ethylsulfonyl-amide and tolylsulfonylamide.

Preferred examples of L and M include hydrogen, hydroxy and oxo and especially, L is hydroxy and M is hydroxy.

Preferred example of A is —COOH, its pharmaceutically acceptable salt, ester or amide thereof.

Preferred example of X₁ and X₂ are hydrogen or halogen, more preferably, both are hydrogen or fluorine atoms at the same time.

Preferred R₁ is a hydrocarbon residue containing 1-10 carbon atoms, preferably 6-10 carbon atoms. Further, at least one carbon atom in the aliphatic hydrocarbon is optionally substituted by oxygen, nitrogen or sulfur.

Examples of R₁ include, for example, the following groups:

### —CH₂—CH₂—CH₂—CH₂—CH₂—CH₂—,

### —CH₂—CH═CH—CH₂—CH₂—CH₂—,

### —CH₂—CH₂—CH₂—CH₂—CH═CH—,

### —CH₂—C≡C—CH₂—CH₂—CH₂—,

### —CH₂—CH₂—CH₂—CH₂—O—CH₂—,

### —CH₂—CH═CH—CH₂—O—CH₂—,

### —CH₂—C≡C—CH₂—O—CH₂—,

### —CH₂CH₂—CH₂—CH₂—CH₂—CH₂—CH₂—,

### —CH₂—CH═CH—CH₂—CH₂—CH₂—CH₂—,

### —CH₂—CH₂—CH₂—CH₂—CH₂—CH═CH—,

### —CH₂—C≡C—CH₂—CH₂—CH₂—CH₂—,

### —CH₂—CH₂—CH₂—CH₂—CH₂—CH(CH₃) —CH₂—,

### —CH₂—CH₂—CH₂—CH₂—CH(CH₃)—CH₂—,

### —CH₂—CH₂—CH₂—CH₂—CH₂—CH₂—CH₂—CH₂,

### —CH₂—CH═CH—CH₂—CH₂—CH₂—CH₂—CH₂—,

### —CH₂—CH₂—CH₂—CH₂—CH₂—CH₂—CH═CH—,

### —CH₂—C≡C—CH₂—CH₂—CH₂—CH₂—CH₂—, and

### —CH₂—CH₂—CH₂—CH₂—CH₂—CH₂—CH(CH₃)—CH₂—.

Preferred Ra is a hydrocarbon containing 1-10 carbon atoms, more preferably, 1-8 carbon atoms. Ra may have one or two side chains each having one carbon atom.

The configuration of the ring and the α- and/or ω chains in the above formula (I) and (II) may be the same as or different from that of the prostanoic acid. However, the present invention also includes a mixture of a compound having a primary type configuration and a compound of a non-primary type configuration.

In this application, a fatty acid derivative wherein the bond between the positions of 13 and 14 is single bond may be in the keto-hemiacetal equilibrium by formation of a hemiacetal between hydroxy at position 11 and keto at position 15.

For example, it has been revealed that when both of X₁ and X₂ are halogen atoms, especially, fluorine atoms, the compound contains a tautomeric isomer, bicyclic compound.

If such tautomeric isomers as above are present, the proportion of both tautomeric isomers varies with the structure of the rest of the molecule or the kind of the substituent present. Sometimes one isomer may predominantly be present in comparison with the other. The fatty acid derivative of the present invention includes both isomers.

Further, the fatty acid derivative used in the invention include the bicyclic compound and analogs or derivatives thereof.

The bicyclic compound is represented by the formula (III):

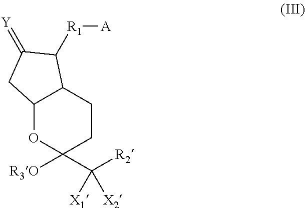

wherein, A is —CH₃, —CH₂OH, —COCH₂OH, —COOH or a functional derivative thereof;

X₁′ and X₂′ are hydrogen, lower alkyl, or halogen;

Y is

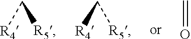

wherein R₄′ and R₅′ are hydrogen, hydroxy, halogen, lower alkyl, lower alkoxy or hydroxy(lower)alkyl, wherein R₄′ and R₅′ are not hydroxy and lower alkoxy at the same time.

R₁ is a saturated or unsaturated bivalent lower or medium aliphatic hydrocarbon residue, which is unsubstituted or substituted with halogen, lower alkyl, hydroxy, oxo, aryl or heterocyclic group, and at least one carbon atom in the aliphatic hydrocarbon is optionally substituted by oxygen, nitrogen or sulfur;

R₂′ is a saturated or unsaturated lower or medium aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; heterocyclic-oxy group; and

R₃′ is hydrogen, lower alkyl, cyclo(lower)alkyl, aryl or heterocyclic group.

While the compounds used in the invention may be represented by a formula or name based on keto-type compound regardless of the presence or absence of the isomers, it is to be noted that such structure or name does not intend to exclude the acetal type compound.

A typical example of the fatty acid derivative in this invention is (Z)-7-[(1R,2R,3R,5S)-3,5-dihydroxy-2-(3-oxodecyl)cyclopentyl]hept-5-enoic acid and its derivatives or analogues. The most favorable example fatty acid derivative in this invention is (+)-isopropyl (Z)-7-[(1R,2R,3R,5S)-3,5-dihydroxy-2-(3-xodecyl)cyclopentyl]hept-5-enoate (hereinafter, isopropyl unoprostone).

In the present invention, any of isomers such as the individual tautomeric isomers, the mixture thereof, or optical isomers, the mixture thereof, a racemic mixture, and other steric isomers may be used in the same purpose.

Some of the compounds used in the present invention may be prepared by the method disclosed in U.S. Pat. Nos. 5,073,569, 5,166,174, 5,221,763, 5,212,324, 5,739,161 and 6,242,485, the contents of these references are herein incorporated by reference.

the fatty acid derivative described as above is useful for treating macular edema. The compound can effectively treat macular edema by administering the same to the patient via non invasive route, for example, ocular topical administration such as instillation of eye drops.

The term “treatment” or “treating” used herein refers to any means of control of a condition including prevention, cure, relief of the condition, attenuation of the condition and arrest of progression.

The macular edema to be treated by the present invention refers to the condition wherein fluid accumulate in the macula and the macular swells, irrespective of the etiology. Diagnosis of macular edema may be carried out by observing the swelling or edema of the retina by means of optical coherence tomography (OCT) and the like.

The concentration of the fatty acid derivative used in the present invention varies depending on the compounds used, kinds of subjects, age, body weight, symptoms to be treated, desired therapeutic effect, dose, treatment duration and the like, and appropriately proper concentration can be selected.

According to the present invention, the fatty acid derivative may be administered systemically or topically. In general, the fatty acid derivative may be administered by ocular topical administration, oral administration, intranasal administration, buccal administration or inhalation. According to the present invention, the fatty acid derivative may preferably be formulated as a pharmaceutical composition suitable for the desired administration by a conventional procedure to provide said composition. The pharmaceutical composition may be those suitable for ocular topical administration, oral administration, intranasal administration, inhalation, intravenous administration including intravenous drip injection, subcutaneous administration and infusion, rectal administration, vaginal administration, or transdermal administration.

The pharmaceutical composition of the present invention may further contain physiologically acceptable additives. Examples of the additive include components used together with the compound of the present invention, such as excipients, diluents, extenders, solvents, lubricants, adjuvants, binders, disintegrants, coating agents, encapsulating agents, ointment bases, suppository bases, aerosols, emulsifiers, dispersing agents, suspending agents, thickeners, isotonizing agents, buffers, analgesics, preservatives, antioxidants, taste adjusting agents, aromatics, coloring materials, functional substances (for example, cyclodextrine, biodegradable polymers, etc.), stabilizer and the like. These additives are well known to a person with an ordinary skill in the art, and may be selected from those described in reference books of general pharmaceutics.

The amount of the above-defined the fatty acid derivative in the pharmaceutical composition of the present invention may vary depending on the formulation of the composition and can generally be within a range of 0.001-10.0 w/v %, more preferably 0.001-5.0 w/v %, and most preferably 0.01-1 w/v %.

Examples of the solid composition for oral administration include tablets, troches, sublingual tablets, capsules, pills, powders, granules and the like. The solid composition may be prepared by mixing one or more active ingredients with at least one inert diluent. The composition may further contain additives other than the inert diluent, for example, lubricants, disintegrants and stabilizers. Tablets and pills may be optionally coated with an enteric-coated or gastric-soluble film. They may be coated with two or more layers. They may be absorbed in a sustained release substance, or microcapsulated. Furthermore, the present composition may be capsulated using an easily decomposable substance such as gelatin. They may be further dissolved in a proper solvent such as fatty acid or a mo-, di- or triglyceride thereof to obtain a soft capsule. In case quick efficacy is required, sublingual tablets may be used.

Examples of the liquid composition for oral administration include emulsions, solutions, suspending agents, syrups, elixirs and the like. The composition may further contain a conventionally used inert diluent, for example, purified water or ethyl alcohol. This composition may contain additives other than the inert diluent, for example, adjuvants such as humectants and suspending agents, sweeteners, flavoring agents, aromatics, preservatives and the like.

The pharmaceutical composition of the present invention may be in the form of a spray composition containing one or more active ingredients, which can be prepared by a known method.

Examples of the intranasal formulation can include aqueous, or oily solutions, suspending agents or emulsions each containing one or more active ingredients. In administration by inhalation of active ingredients, the composition of the present invention can be in the form of a suspension, solution or emulsion capable of providing as an aerosol, or in the form of a powder suited for inhalation of a dry powder. The composition for administration by inhalation can further contain propellants which are commonly used.

Examples of the injection composition for parenteral administration of the present invention can include sterilized aqueous or non-aqueous solutions, suspending agents, emulsions and the like. Examples of the diluent for aqueous solutions or suspending agents include distilled water for injection, physiological saline, Ringer's solution and the like.

The non-aqueous diluent for solutions and suspending agents can include, for example, propylene glycol, polyethylene glycol, vegetable oils (olive oil, etc.), alcohols (ethanol, etc.), polysorbates and the like. This composition may further contain additives such as preservatives, humectants, emulsifier and dispersing agents. The composition may be sterilized, for example, by filtering through a bacteria reservation filter, blending a sterilizing agent, or a gas or radioisotope radiation sterilization. The composition for injection can be provided as sterilized powder composition, or can be dissolved in a sterilized solvent for injection before use.

An external use medicine of the present invention includes any external formulation used in the fields of dermatology and otolaryngology, and examples thereof include ointments, creams, lotions, sprays and the like.

The pharmaceutical composition of the present invention may include suppositories or pessaries, and these can be usually prepared by mixing a commonly used base, for example, cocoa butter which is softened at body temperature, with an active ingredient and a nonionic surfactant having a proper softening temperature suited for an improvement of absorbency may also be used.

In the present invention, the fatty acid derivative may preferably be formulated into an ophthalmic composition and is topically administered to the eyes of the patient. The ophthalmic composition of the present invention includes any dosage form for ocular topical administration used in the field of ophthalmology, such as an ophthalmic solution, an eye drop and an eye ointment. The ophthalmic composition can be prepared in accordance with conventional means known in the relevant technical field.

The ophthalmic solution or eye drop may be prepared by dissolving the active ingredient in a solvent such as an aqueous sterilization solution (for example, brine and buffered solution). The ophthalmic composition may also be prepared as a powder composition comprising the active ingredient which is dissolved at the time of use. The ophthalmic solution of the present invention may further comprises additives which have been employed in conventional ophthalmic solutions such as buffers and isotonic agents.

An isotonic agent may be any one used usually in the ophthalmology field. Examples of the isotonic agents include, but are not limited to, sodium chloride, potassium chloride, calcium chloride, sodium hydrogen carbonate, sodium carbonate, magnesium sulfate, sodium hydrogen phosphate, sodium dihydrogen phosphate, potassium dihydrogen phosphate, boric acid, borax, sodium hydroxide, hydrochloric acid, mannitol, sorbitol, glucose, glycerin, propylene glycol, polyethylene glycol and the like. The isotonic agent may preferably be a sugar alcohol such as mannitol or sorbitol and/or a polyol such as glycerin or propylene glycol.

In the present invention, in order to improve solubility of the fatty acid derivative in the solvent, a solubilizing agent such as a surfactant can be used. The surfactant used in the present invention is not limited as long as it can achieve the object, and a nonionic surfactant is preferred. Examples of the nonionic surfactant include polyoxyethylene sorbitan fatty acid esters such as polyoxyethylene sorbitan monooleate (Polysorbate 80), polyoxyethylene sorbitan monostearate (Polysorbate 60), polyoxyethylene sorbitan monopalmitate (Polysorbate 40), polyoxyethylene sorbitan monolaurate, polyoxyethylene sorbitan trioleate and polyoxyethylene sorbitan tristearate (Polysorbate 65); polyoxyethylene hardened castor oils such as polyoxyethylene hardened castor oil 10, polyoxyethylene hardened castor oil 40, polyoxyethylene hardened castor oil 50 and polyoxyethylene hardened castor oil 60; polyoxyethylene polyoxypropylene glycols such as polyoxyethylene (160) polyoxypropylene (30) glycol [Pluronic F68] and polyoxyethylene (42) polyoxypropylene (67) glycol [Pluronic P123]; polyoxyethylene fatty acid esters such as polyoxyethylene 40 monostearate; and polyoxyethylene alkyl ethers such as polyoxy 10 oleyl ether (Brij 97) and polyoxyl 20 oleyl ether (Brij 98). Preferably, polyoxyethylene sorbitan monooleate (Polysorbate 80), polyoxyethylene hardened castor oil 60, polyoxyethylene 40 monostearate, polyoxyl 10 oleyl ether and the like are exemplified, and these nonionic surfactants may be used alone, or two or more kinds of them may be used in combination.

Furthermore, additive used usually in the field of ophthalmology may be optionally added to the composition of the present invention. Examples of the additive include buffers (for example, boric acid, borax, sodium hydrogen phosphate and sodium dehydrogen phosphate, sodium edetate), preservatives (for example, benzalkonium chloride, benzethonium chloride and chlorobutanol), thickeners (for example, polysaccharides such as sodium hyaluronate, chondroitin sulfate, guar gum, gellan gum, xantan gum and sodium alginate; cellulose polymers such as methyl cellulose, methyl ethyl cellulose and hydroxypropyl methyl cellulose; sodium polyacrylate, a carboxyvinyl polymer and a crosslinked polyacrylic acid.

The eye ointment is prepared by mixing an active ingredient with an ointment base. Examples of the eye ointment bases include, but are not limited to, oily bases such as petrolatum, liquid paraffin, polyethylene, Selene 50, Plastibase, macrogol or a combination thereof; emulsion bases containing an oil phase and an aqueous phase emulsified by the surfactant; and water-soluble bases such as hydroxypropyl methyl cellulose, carboxypropyl methyl cellulose and polyethylene glycol.

The composition of the present invention may be formulated as a sterile unit dose containing no preservative.

In the present invention, in the case of using isopropyl unoprostone, the concentration of the compound is 0.12 w/v % or more, and preferably 0.15 w/v % or more. The upper limit of the concentration is not particularly restrictive and may be set at approximately 10 w/v %.

The method of administrating the ophthalmic composition used in the present invention varies depending on the compounds used, kinds of subject such as animals or humans, age, body weight, symptoms to be treated, desired therapeutic effect, treatment duration and the like. In the case of an ophthalmic solution or eye drop, at least three or more drops may be administered per day. Regarding timing of administration, it is possible to administer with a given interval (for example, every 5 hours) or to administer continuously. In the case of two or more drops per one administration are instilled to one eye, one drop is preferably administered with at least 5 minute interval after the instillation of the previous drop. Preferred dosage regimen includes instillation of at least four or more drops per day. The dosage regimen can be achieved by instilling two or more drops per one administration, twice or more times a day. In this dosage regimen, the second drop is instilled 5 minutes after the instillation of the first drop. In case the number of drops further increases, each drop can also be instilled every 5 minutes. The number of administrations per day is from approximately 2 to 12 times, and the number of drops per one time administration is from two drops to approximately twelve drops.

The one drop volume of the ophthalmic composition used in the present invention may be at least approximately 20 μL or more, preferably approximately 30 μL or more, usually approximately from 20 to 50 μL, and preferably approximately from 30 to 40 μL. In the case of using the ophthalmic solution or eye drop of isopropyl unoprostone (0.12 w/v %), one drop of approximately 20 μL comprises approximately 24 μg of the active compound. When three drops per day are instilled, the total daily dose of the active compound will be approximately 72 μg and when four drops, the total daily dose will be approximately 96 μg. In the case of using the ophthalmic solution or eye drop of isopropyl unoprostone ester (0.15 w/v %), one drop of approximately 20 μL comprises approximately 30 μg of the active compound. When three drops per day are instilled, the total daily dose of the active compound will be approximately 90 μg and when four drops, the total daily dose will be approximately 140 μg. When the volume of one drop is approximately 30 μL, the amount of the active compound per one drop is approximately 45 μg. When three drops per day are instilled, the total daily dose of the active compound will be approximately 135 μg and when four drops, the total daily dose will be approximately 180 μg.

The term “approximately” used herein can mean plus or minus a range of up to 30%, preferably up to 20%, more preferably up to 10%.

In order to achieve an object of the present invention, the dose of the ophthalmic solution or eye drop per se to be administered per one eye per day may also be increased as compared with the dose based on the application of the fatty acid derivative typified by isopropyl unoprostone to glaucoma. Therefore, in order to solve the problem of the side effect due to antiseptics such as benzalkonium chloride, an ophthalmic composition substantially free from benzalkonium chloride is preferred in the present invention.

In the present specification, the term “ophthalmic composition substantially free from benzalkonium chloride” means that the composition contains no benzalkonium chloride or the composition contains a given concentration or less of benzalkonium chloride. In the present invention, the concentration of benzalkonium chloride of the ophthalmic composition is less than 0.01 w/v %, preferably 0.005 w/v % or less, and more preferably 0.003 w/v % or less. Also, using a sterile unit dose formulation (for example, one-day disposable or a single dose unit) free from a preservative such as benzalkonium chloride is one of preferred means of the present invention.

It is surprising that the fatty acid derivative having an oxo group at position 15 of prostanoic acid skeleton such as isopropyl unoprostone could effectively treat macular edema while latanoprost, which is a fatty acid derivative having a hydroxy group at position 15 of prostanoic acid skeleton, causes macular edema as a side effect.

That is, a pharmaceutical composition comprising the fatty acid derivative of the present invention is useful for the treatment of macular edema.

The present invention will be described in more detail by way of Examples, but the present invention is not limited thereto.

### EXAMPLES

### Formulation Example 1

The respective components were dissolved in purified water so as to adjust to each w/v % shown below, and the solution was aseptically filtered and then filled into a sterilized low density polyethylene container to obtain an ophthalmic solution (one drop volume: approximately 35 μL).

| | |
|---|---|
| 0.15% | isopropyl unoprostone |
| 1.0% | Polyoxyethylene sorbitan monooleate |
| 1.0% | Mannitol |
| 1.9% | Glycerin |
| 0.05% | Sodium edetate |
| 0.003% | Benzalkonium chloride |

### Formulation Example 2

Using the solution prepared by dissolving the respective components in purified water so as to adjust to each w/v % shown below and aseptically filtrating, a sterile unit dose (one-day disposable type) ophthalmic solution was obtained by a Blow Fill Seal system.

| | |
|---|---|
| 0.18% | isopropyl unoprostone |
| 0.70% | Polyoxyethylene sorbitan monooleate |
| 0.30% | Polyoxyl 10 oleyl ether |
| 4.7% | Mannitol |
| 0.01% | Sodium edetate |

### Formulation Example 3

Using the solution prepared by dissolving the respective components in purified water so as to adjust to each w/v % shown below and aseptically filtrating, a sterile unit dose (single unit dose type) ophthalmic solution was obtained by a Blow Fill Seal system.

| | |
|---|---|
| 0.24% | isopropyl unoprostone |
| 0.95% | Polyoxyethylene sorbitan monooleate |
| 0.42% | Polyoxyl 10 oleyl ether |
| 4.7% | Mannitol |
| 0.01% | Sodium edetate |
| 0.02% | Borax |
| 0.05% | Sodium edentate |
| 0.6% | Xanthan gum |

### Test Example 1

The ophthalmic solution of formulation Example 1 was administered to a patient with retinitis pigmentosa associated with a complication of mild macular edema.

Patient (female, aged 49) at 0 week
Retinitis Pigmentosa (idiopathic)
Complication: mild macular edema (both eyes), cataract (both eyes)
Goldman visual field test: late stage retinitis pigmentosa (both eyes)

The ophthalmic solution was instilled two drops per one time administration (with 5 minute interval), twice a day for 24 weeks. Before (0 week) and after (24 weeks) the treatment, macular of the patient's eye were evaluated. The evaluation was performed by confirming the presence or absence of edema or cyst using an optical interference tomograph (OCT). The results are shown in Table 1.

TABLE 1
Findings of OCT (optical interference tomograph)

Edema or Cyst

| Eye to be evaluated | Right eye | Left eye |
|---|---|---|
| Pre-observation | Observed | Observed |
| period (0 week) | | |
| Treatment completion | Not observed | Not observed |
| period (24 weeks) | | |

An improvement in macular edema was recognized by the instillation of two drops per time administration, twice daily. As is apparent from the result, the composition of the present invention can treat macular edema by ocular topical administration.

### Test Example 2

The effect of the fatty acid derivative to inhibit retinal vascular leakage in rabbit model.

Animals: GD79B rabbit (pigmented). Each group contains 8 animals.

### Test Compositions:

1) 0.15 w/v % isopropyl unoprostone: formulation example 1
2) 4 w/v % triamcinolone acetonide: Kenacort® Retard (Bristol-Myers Squibb Japan, Tokyo, JP) (positive control)
3) vehicle of isopropyl unoprostone (composition comprising the ingredients of formulation example 1 except for isopropyl unoprostone) (control)

Retinal vascular leakage model animals were prepared. Recombinant human vascular endothelial cell growth factor (rhVEGF) 500 ng (in 50 μl) was injected intravitreally in the right eye of animals. Just after the injection of rhVEGE, 50 μl of test composition 1 or 2 was injected intraviterally in the right eye of the animal. The left eyes were remain untreated (no rhVEGE nor test compositions).

Forty seven (47) hours after the rhVEGF injection, 50 mg/kg of sodium fluorescein (10 w/v % in physiological saline) was injected to the animals via auricular veins. One (1) hour after the injection of sodium fluorescein, the fluorescence intensities in the vitreous body of the treated and untreated eyes were measured by using a corneal spectrophotometer. Based on the measured value, area under the curve (AUC) of the fluorescence intensity that reflects the leakage and the ratio of AUC (treated eye/untreated eye, i.e. right eye/left eye) were calculated. The AUC ratio of isopropyl unoprostone and triamcinolone acetonide administered group were compared to the AUC ratio of the control group to give the inhibition (%) of retinal vascular leakage.

Result is shown in Table 3 below. In the isopropyl unoprostone group, fluorscein leakage was inhibited by 60%. In the triamcinolone acetonide group (positive control group), fluorescein leakage was inhibited by 89%. This results indicate that isopropyl unoprostone can inhibit retinal vascular leakage or vascular hypermeability due to the degradation of the blood-retinal barrier and hence, is useful for the treatment of macular edema.

TABLE 3
Inhibition of hrVEGF induced retinal vascular leakage

| | Fluorescein Leakage (AUC) | | | | | |
|---|---|---|---|---|---|---|
| | right eye | | left eye | | ratio of AUC | Inhibition of |
| | (treated) | | (non-treated) | | right/left | fluorescein leakage |
| group | Mean | S.D. | Mean | S.D. | Mean ± S.D. | (vs. control) |
| 0.15 w/v % | 10998.4 | 11301.9 | 3302.1 | 794.7 | 3.43 ± 4.01 | 60% |
| isopropyl | | | | | | |
| unoprostone | | | | | | |
| 4 w/v % | 3715.6 | 1576.8 | 3997.4 | 1296.6 | 0.98 ± 0.44 | 89% |
| triamcinolone | | | | | | |
| acetonide | | | | | | |

What is claimed is:

1. A method for treating macular edema in a mammalian subject, comprising administering an effective amount of a fatty acid derivative represented by the formula (I): 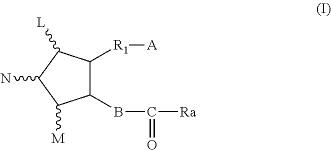
wherein L, M and N are hydrogen, hydroxy, halogen, lower alkyl, hydroxy(lower)alkyl, lower alkanoyloxy or oxo, wherein at least one of L and M is a group other than hydrogen and the five-membered ring may have at least one double bond;
A is —CH₃, —CH₂OH, —COCH₂OH, —COOH or a functional derivative thereof;
B is single bond, —CH₂—CH₂—, —CH═CH—, —C≡C—, —CH₂—CH₂—CH₂—, —CH═CH—CH₂—, —CH₂—CH═CH—, —C≡C—CH₂— or —CH₂—C≡C—;
R₁ is saturated or unsaturated bivalent lower or medium aliphatic hydrocarbon residue, which is unsubstituted or substituted with halogen, lower alkyl, hydroxy, oxo, aryl or heterocyclic group, and at least one of carbon atom in the aliphatic hydrocarbon is optionally substituted by oxygen, nitrogen or sulfur; and
Ra is saturated or unsaturated lower or medium bivalent aliphatic hydrocarbon residue, which is unsubstituted or substituted with halogen, oxo, hydroxy, lower alkyl, lower alkoxy, lower alkanoyloxy, cyclo(lower)alkyl, cyclo(lower)alkyloxy, aryl, aryloxy, heterocyclic group or hetrocyclic-oxy group; lower alkoxy; lower alkanoyloxy; cyclo(lower)alkyl; cyclo(lower)alkyloxy; aryl; aryloxy; heterocyclic group; or heterocyclic-oxy group;
to the subject in need thereof.

2. The method of claim 1, wherein Ra is a hydrocarbon containing 6-10 carbon atoms.

3. The method of claim 2, wherein Ra is a hydrocarbon containing 7 carbon atoms.

4. The method of claim 1, wherein L is hydroxy, M is hydrogen and N is hydroxy.

5. The method of claim 4, wherein R₁ is —CH₂—CH═CH—CH₂—CH₂—CH₂— and Ra is a hydrocarbon containing 7 carbon atoms.

6. The method of claim 1, wherein B is —CH₂—CH₂—.

7. The method of claim 6, wherein the fatty acid derivative is isopropyl unoprostone.

8. The method of claim 1, wherein the fatty acid derivative is administered topically to the eyes.

9. The method of claim 8, wherein an ophthalmic solution comprising the fatty acid derivative is instilled to the eyes.
